(19) United States
(12) Patent Application Publication
(10) Pub. No.: US 2015/0325137 A1
(43) Pub. Date: Nov. 12, 2015

(54) METHOD AND SYSTEM FOR A COGNITIVE TRAINING PROGRAM

(71) Applicant: Home Care Assistance, Inc., Palo Alto, CA (US)

(72) Inventors: James Harding Johnson, Palo Alto, CA (US); Samuel Thomas Gontkovsky, San Francisco, CA (US)

(73) Assignee: Home Care Assistance, Inc., Palo Alto, CA (US)

(21) Appl. No.: 14/274,076

(22) Filed: May 9, 2014

(51) Int. Cl. G09B 19/00; A61B 5/16

(52) U.S. Cl. CPC G09B 19/00; A61B 5/16

(57) ABSTRACT

A method for managing intervention activities for chronic progressive neurodegenerative conditions that enhance neurophysiological processes of a client includes receiving a client for evaluation and determining a first level of functioning in each of a plurality of cognitive and non-cognitive areas. The method also includes iteratively, determining a program of intervention activities associated with the cognitive and non-cognitive areas based on at least one of the first level of functioning and a generated report of a performance of the intervention activities, the program of intervention activities including one or more intervention activities selected from a plurality of intervention activities, administering the program of intervention activities to the client, and generating a report of a performance of the administered program of intervention activities. The method further includes reporting a progress of the client over time with respect to the performance of the administered program of intervention activities.

### BACKGROUND OF THE DISCLOSURE

This description relates to cognitive training, and, more particularly, to a method and system for generating and administering a personalized training program for an individual based on a comprehensive evaluation process.

Dementia is a serious loss of cognitive ability in a previously-unimpaired person, beyond what might be expected from normal aging. It may be static, the result of a unique global brain injury, or progressive, resulting in long-term decline due to damage or disease in the body. Although dementia is far more common in the geriatric population, it may occur in any stage of adulthood. Dementia which begins gradually and worsens progressively over several years is usually caused by neurodegenerative disease, that is, by conditions affecting only or primarily the neurons of the brain and causing gradual but irreversible loss of function of these cells.

Dementia is a non-specific illness syndrome in which affected areas of cognition may be memory, attention, language, visual-spatial perception, and executive functioning, as well as basic and instrumental activities of daily living (ADLs), social skills, and behavioral disturbances. Especially in the later stages of the condition, affected persons may be disoriented in time (not knowing what day of the week, day of the month, or even what year it is), in place (not knowing where they are), and in person (not knowing who they are or others around them). The causes of dementia depend on the age at which symptoms begin. In the elderly population (usually defined in this context as over 65 years of age), a large majority of cases of dementia are caused by Alzheimer's disease and vascular dementia. Dementia with Lewy bodies is another fairly common cause, which again may occur alongside either or both of the other causes. Hypothyroidism sometimes causes slowly progressive cognitive impairment as the main symptom, and this may be fully reversible with treatment. Normal pressure hydrocephalus, though relatively rare, is important to recognize since treatment may prevent progression and improve other symptoms of the condition. However, significant cognitive improvement is unusual.

Various types of brain injury, occurring as a single event, may cause irreversible but fixed cognitive impairment. Traumatic brain injury may cause generalized damage to the white matter of the brain, or more localized damage. A temporary reduction in the brain's supply of blood or oxygen may lead to hypoxic-ischemic injury. Strokes (ischemic stroke, or intracerebral, subarachnoid, subdural or extradural hemorrhage) or infections (meningitis and/or encephalitis) affecting the brain, prolonged epileptic seizures and acute hydrocephalus may also have long-term effects on cognition. Excessive alcohol use may cause either alcohol dementia or Korsakoff s psychosis (and certain other recreational drugs may cause substance-induced persisting dementia); once overuse ceases, the cognitive impairment is persistent but non-progressive.

Alzheimer's disease (AD), also called Alzheimer disease, senile dementia of the Alzheimer Type (SDAT) or simply Alzheimer's, is the most common form of dementia. It is an incurable, degenerative, and terminal disease and is generally diagnosed in people over 65 years of age, although the less-prevalent early-onset Alzheimer's can occur much earlier. In 2006, there were 26.6 million sufferers worldwide, with about ⅓ of people over the age of 80 suffering from it. Alzheimer's is predicted to affect 1 in 85 people globally by 2050. Public expenditure on AD is overwhelming, reaches nearly $100B per annum in the United States alone, and over $250B per annum in the largest seven western countries. Although the course of Alzheimer's disease is unique for every individual, there are many common symptoms. In the early stages, the most commonly recognized symptom is memory loss, such as difficulty in remembering recently learned facts. Diagnosis is usually confirmed with behavioral assessments and cognitive tests, sometimes followed by a brain scan. As the disease advances, symptoms include confusion, irritability and aggression, mood swings, language breakdown, long-term memory loss, and the general withdrawal of the sufferer as their senses decline. Gradually, bodily functions are lost, ultimately leading to death.

Currently used treatments offer only a minor symptomatic benefit; no treatments significantly delaying (beyond 3-6 months) or halting the progression of the disease are as yet available. Mental stimulation, exercise, and a balanced diet have been suggested, as both a possible prevention and a sensible way of managing the disease. Most patients today receive drugs of the family choline-esterase-inhibitors (ChEI), which increase the concentration in the brain of acetyl-choline. However, some do not see any improvement from the drug, and for others improvement caused by the drug lasts between approximately three and twelve months, and then the normal degradation of the patient resumes. Other treatments being attempted include magnetic stimulation of selected regions of the brain.

### BRIEF DESCRIPTION OF THE DISCLOSURE

In one aspect, a method for managing intervention activities for chronic progressive neurodegenerative conditions that enhance neurophysiological processes of a client includes receiving a client for evaluation and determining a first level of functioning in each of a plurality of cognitive and non-cognitive areas. The method also includes iteratively, determining a program of intervention activities associated with the cognitive and non-cognitive areas based on at least one of the first level of functioning and a generated report of a performance of the intervention activities, the program of intervention activities including one or more intervention activities selected from a plurality of intervention activities, administering the program of intervention activities to the client, and generating a report of a performance of the administered program of intervention activities. The method further includes reporting a progress of the client over time with respect to the performance of the administered program of intervention activities.

In another aspect, a method of enhancing neurophysiological processes of a client includes receiving a program of intervention activities including a plurality of intervention activities, each intervention activity associated with at least one of a cognitive and a non-cognitive area and administering at least a portion of the program of intervention activities to the client, the portion based on a level of cooperation of the client. The method also includes recording an evaluation of the success of the administering based on predetermined completion thresholds and reporting a progress of the client over time with respect to the recorded evaluations for each of the domains, cognitive subdomains, and non-cognitive areas evaluated.

In yet another aspect, a method of administering a program of intervention activities to reduce progress of a chronic progressive neurodegenerative condition includes receiving an evaluation of a client, the evaluation including at least one of a new client evaluation and a progress evaluation, receiving a plurality of available intervention activities from a library of approved intervention activities, and generating a program of intervention activities for administration to the client based on the at least one of a new client evaluation and a progress evaluation, and the plurality of available intervention activities, the program of intervention activities including a plurality of the available intervention activities, each intervention activity in the plurality of the available intervention activities being associated with a domain, a cognitive subdomain, and/or a non-cognitive area. The method also includes transmitting the program of intervention activities to the staff personnel for administration to the client and reporting the progress of the client.

### BRIEF DESCRIPTION OF THE DRAWINGS

FIGS. 1-4 show example embodiments of the method and apparatus described herein.

FIG. 1 is a data flow diagram of a method for managing a program of intervention activities for chronic progressive neurodegenerative conditions.

FIG. 2 illustrates example panels of interventions designed to stimulate, for example, one or more of the five primary cognitive subdomains.

FIG. 3 illustrates example panels of interventions designed to stimulate, for example, one or more of the non-cognitive areas.

FIG. 4 is a flowchart of an example method for managing intervention activities for chronic progressive neurodegenerative conditions that enhance neurophysiological processes of a client.

Although specific features of various embodiments may be shown in some drawings and not in others, this is for convenience only. Any feature of any drawing may be referenced and/or claimed in combination with any feature of any other drawing.

Unless otherwise indicated, the drawings provided herein are meant to illustrate features of embodiments of the disclosure. These features are believed to be applicable in a wide variety of systems comprising one or more embodiments of the disclosure. As such, the drawings are not meant to include all conventional features known by those of ordinary skill in the art to be required for the practice of the embodiments disclosed herein.

### DETAILED DESCRIPTION OF THE DISCLOSURE

The following detailed description illustrates embodiments of the disclosure by way of example and not by way of limitation. It is contemplated that the disclosure has general application to analytical and methodical embodiments of administering and managing a plurality of selected interventions to a client based at least in part on a comprehensive evaluation and information gathering process.

Embodiments of the disclosure describe a cognitive training program for certain clients. Clients may be those affected by symptoms of a loss of cognitive ability or those who have not yet experienced symptoms of the loss of cognitive ability, but who may be at risk through family history, or medical or genetic testing. The in-home program is administered to clients experiencing cognitive difficulties ranging in degree from no outward symptoms, mild cognitive impairment, or more severe symptoms.

The program is highly personalized, and activities are created for each client based on an evaluation process. The evaluation utilizes a proprietary behavioral rating scale and is completed by a family member of the client. It is used to measure any changes against a re-evaluation when it is completed. In various embodiments, the evaluation includes a behavior rating index, which is an index questionnaire that a family member of the client completes in a hard copy format or an online format. A scale is used to rate the client's behavior, responsiveness, or frequency of certain actions on a scale, such as, but, not limited to “never” to “always.” In an embodiment, there are more than twenty actions that are evaluated for the index. Such behavior includes, does the client seem angry or frustrated? Does the client initiate conversation?

Training activities are administered by trained staff personnel to provide cognitive stimulation to clients. The trained staff personnel complete extensive training to administer activities chosen from over three-hundred interventions representing seven domains. The seven domains include a cognitive domain, a coping domain, a social domain, a recreational domain, an exercise domain, a sensory domain, and a diet domain. The cognitive subdomain includes five subdomains; memory, executive functioning, attention, language and visual spatial perception. An intervention plan is adapted and revised according to the client's abilities, limitations, personal interests, preferences and histories after the admission evaluation is completed. The interventions address a wide array of cognitive domains and non-cognitive areas that are not limited to memory.

The interventions target five primary cognitive subdomains that have been shown to be affected by dementia, as well as non-cognitive areas. Executive functioning includes cognitive abilities such as reasoning, problem solving, judgment, and cognitive flexibility. Attention refers to the ability to focus on a specific piece of information for a sustained period of time while suppressing awareness of other competing distractions. Language refers to the ability to execute verbal functions including spontaneous speech, speech repetition, speech comprehension, naming, reading and writing. Visual-spatial perception involves the ability to accurately perceive and understand the visual relationships between objects and space. Memory refers to the ability to retain information and utilize it later for adaptive purposes.

As used herein, “cognitive training” refers to any non-pharmacological intervention designed to improve cognition, regardless of mechanism of action. Typically, cognitive training focuses on specific cognitive domains or cognitively mediated domains of functioning, such as basic and instrumental activities of daily living (ADLs), social skills, and behavioral disturbances. Cognitive training includes cognitive stimulation, memory rehabilitation, reality orientation, and neuropsychological rehabilitation.

As used herein, cognition is a general term for mental processes by which an individual acquires knowledge, solves problems, and plans activities. Cognitive skills include memory, executive functioning, attention, language and visual spatial perception, among others. The functional levels of each of these skills can be studied alone or in combination for a particular individual.

The following description refers to the accompanying drawings, in which, in the absence of a contrary representation, the same numbers in different drawings represent similar elements.

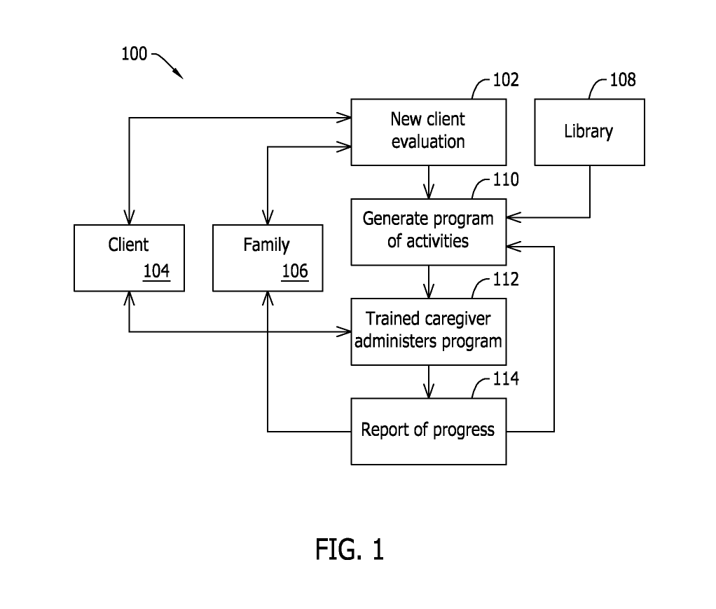

FIG. 1 is a data flow diagram of a method 100 for managing a program of intervention activities for chronic progressive neurodegenerative conditions. In the example embodiment, method 100 includes receiving a new client 104 for a new client evaluation 102. New client evaluation 102 includes a questionnaire type format that is completed by family member 106 and an assessment form administered by staff 112 to client 104. The results of new client evaluation 102 and a library 108 of intervention activities are used to generate the program of intervention activities 110. Program of intervention activities 110 includes intervention activities that have been developed by a research and operations team (not shown) as activities determined to stimulate at least one of the domains, subdomains, and non-cognitive areas. The selected intervention activities that are included in the program are made available to the staff for administration 112 to the client during a session in the client's usual living space. During the administration 112 of the selected intervention activities, the staff evaluates and reports on the cooperation, engagement, and performance of the client with respect to the selected intervention activities.

A report of progress 114 is generated by the staff during and after administration 112. Report of progress is used to maintain family 106 informed of the progress of client 104 and also to provide input for the research and operations team for modifying the program of activities 110 for client 104. Method 100 continues until a determination is made that method 100 is no longer beneficial to client 104. For example, if the client's condition advances to the point where client 104 is no longer cooperative, method 100 will end for that client. Even if client 104 is unable to accurately complete the selected intervention activities, method 100 can continue for client 104, if client 104 is willing to and has the ability to participate in the activities.

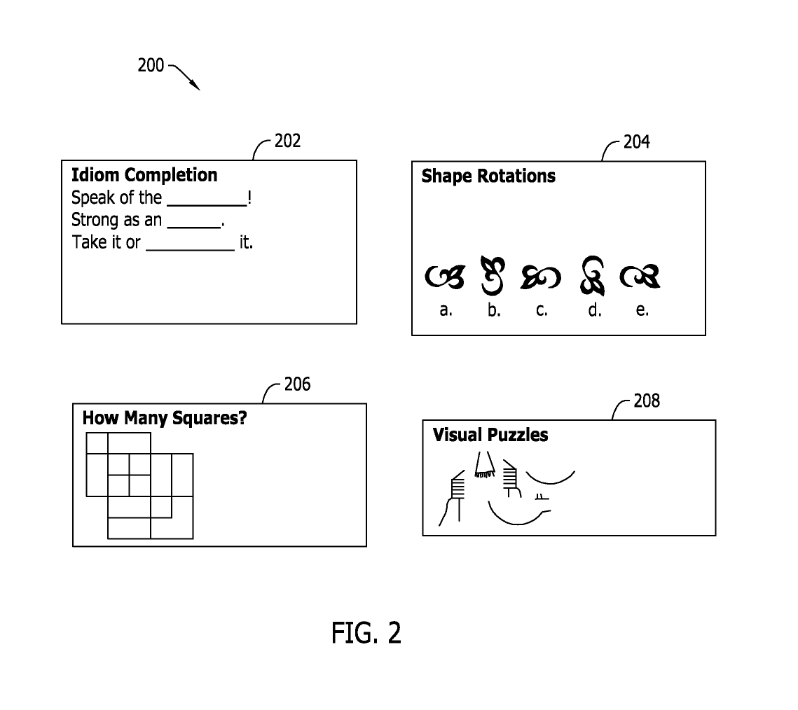

FIG. 2 illustrates example panels 200 of interventions designed to stimulate, for example, one or more of the five primary cognitive subdomains. A first panel 202 illustrates intervention activities for completing common idioms. Idiom completion targets the language primary cognitive subdomain. A second panel 204 illustrates shape rotations, which target the visual-spatial perception subdomain. A third panel 206 illustrates intervention activities for determining relationships among geometric shapes, which also targets the visual-spatial perception subdomain. A fourth panel 208 illustrates intervention activities for rearranging shapes to reform familiar objects, which also targets the visual-spatial perception primary cognitive subdomains.

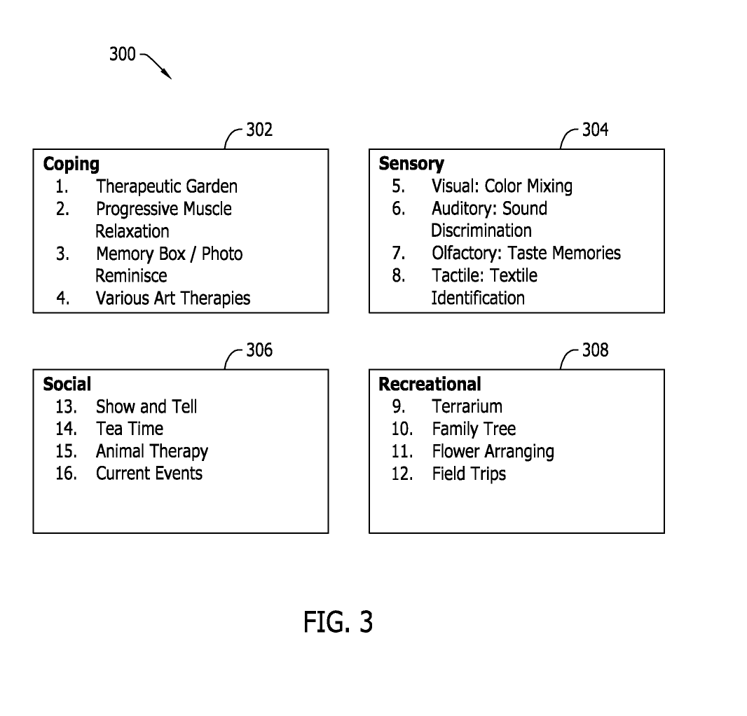

FIG. 3 illustrates example panels 300 of interventions designed to stimulate, for example, one or more of the non-cognitive areas. A first panel 302 illustrates intervention activities for the coping domain. For example, a therapeutic garden may facilitate reliving stress in client 104. Progressive muscle relaxation and a memory box or photo reminiscence album may be included in the intervention activities to promote relaxation, and happiness. Various art therapies facilitate stimulating creativity and imagination as well as maintaining fine motor control.

A second panel 304 illustrates intervention activities for the sensory domain. For example, the visual sense is stimulated using, for example, color mixing, the auditory sense is stimulated using sound discrimination to exercise client's 104 ability to notice distinct sounds. The olfactory sense is stimulated using taste memories and the tactile sense is stimulated using textile identification exercises.

A third panel 306 illustrates intervention activities for the social domain. For example, activities that encourage communication, introductions to new persons, and animal therapy stimulate the social domain of client 104. Activities such as show and tell and tea time encourage social interaction with others also involved in the activities. Current events discussions also permit stimulation of the social domain.

A fourth panel 308 illustrates intervention activities for the recreational domain. The intervention activities for the recreational domain include hobbies and enjoyable activities, such as, but not limited to maintaining a terrarium, constructing a family tree, flower arranging, and field trips.

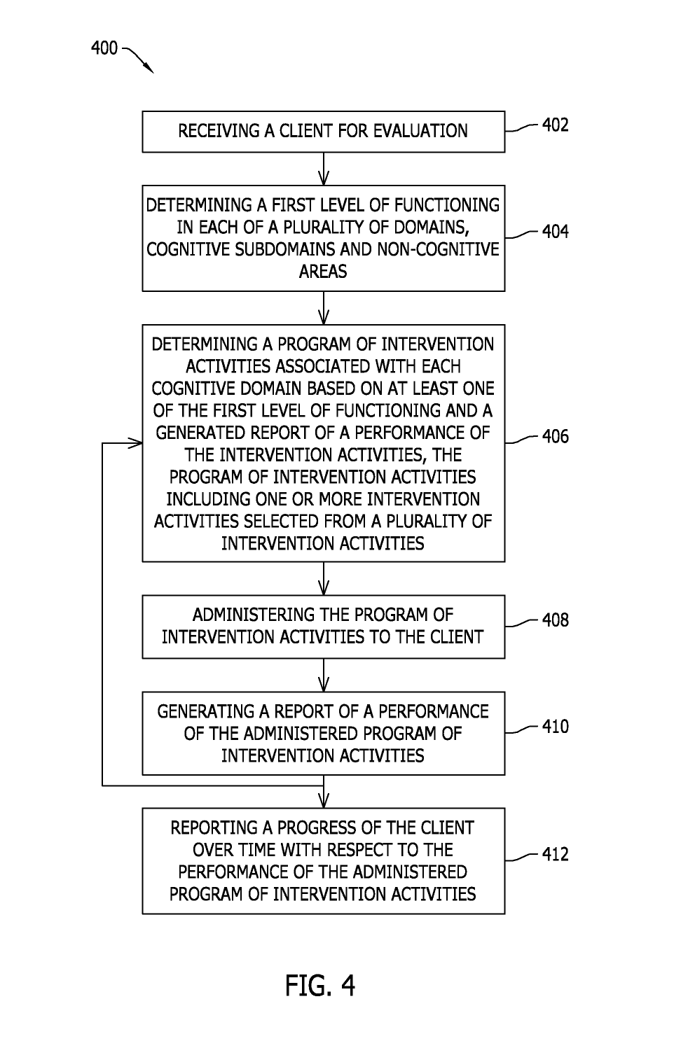

FIG. 4 is a flowchart of an example method 400 for managing intervention activities for chronic progressive neurodegenerative conditions that enhance neurophysiological processes of a client. In the example embodiment, the method includes receiving 402 a client for evaluation, determining 404 a first level of functioning in each of a plurality of domains, cognitive subdomains and non-cognitive areas, and determining 406 a program of intervention activities associated with each cognitive domain based on at least one of the first level of functioning and a generated report of a performance of the intervention activities, the program of intervention activities including one or more intervention activities selected from a plurality of intervention activities. Method 400 further includes administering 408 the program of intervention activities to the client, and generating 410 a report of the administered intervention activities. Method 400 further includes reporting 414 a progress of the client over time with respect to the cooperation, engagement, and performance of the administered program of intervention activities. The determining 406, administering 408, and generating 410 steps are performed iteratively until client 104 is no longer enrolled in method 400. In some iterations, the program of intervention activities may not be altered based on the generating 410 step, but rather, the program may be left unchanged.

Optionally, method 400 also includes generating a program of intervention activities associated with a cognitive domain, a coping domain, a social domain, a recreational domain, an exercise domain, a sensory domain, and a diet domain.

The foregoing detailed description illustrates embodiments of the disclosure by way of example and not by way of limitation. It is contemplated that the disclosure has general application to the evaluation, administration, and management of a regime of interventions to a client. It is further contemplated that the methods and systems described herein may be incorporated into existing healthcare planning systems, in addition to being maintained as a separate stand-alone application.

It will be appreciated that the above embodiments that have been described in particular detail are merely example or possible embodiments, and that there are many other combinations, additions, or alternatives that may be included.

Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

The above-described embodiments of a method and system for providing interventions for chronic progressive neurodegenerative conditions provides a cost-effective and reliable means for managing communications amongst providers and clients, historical recordkeeping, reporting of results and progress, documenting interventions and scores of achievement of each goal of the intervention. In addition, the above-described methods and systems facilitate periodic evaluation of progress and adjusting assigned interventions based on the progress. As a result, the methods and systems described herein facilitate managing interventions in a cost-effective and reliable manner.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for managing intervention activities for chronic progressive neurodegenerative conditions that enhance neurophysiological processes of a client, the method comprising:
receiving a client for evaluation;
determining a first level of functioning in each of a plurality of cognitive and non-cognitive areas;
iteratively,
determining a program of intervention activities associated with the cognitive and non-cognitive areas based on at least one of the first level of functioning and a generated report of a performance of the intervention activities, the program of intervention activities including one or more intervention activities selected from a plurality of intervention activities;
administering the program of intervention activities to the client; and
generating a report of a performance of the administered program of intervention activities; and
reporting a progress of the client over time with respect to the performance of the administered program of intervention activities.

2. The method of claim 1, wherein receiving a client for evaluation comprises receiving a client that is asymptomatic to a chronic progressive neurodegenerative condition.

3. The method of claim 1, wherein determining a first level of functioning in each of a plurality of cognitive subdomains comprises determining a first level of functioning in each of a memory subdomain, an executive function subdomain, an attention subdomain, a visual-spatial subdomain, and a language subdomain.

4. The method of claim 1, wherein determining a first level of functioning in each of a plurality of cognitive and non-cognitive areas comprises determining a first level of functioning by direct observation of at least one of a family member and a staff.

5. The method of claim 4, wherein determining a first level of functioning by direct observation of at least one of a family member and a staff comprises subjectively scoring the first level of functioning on a predetermined scale.

6. The method of claim 1, wherein determining a first level of functioning in each of a plurality of cognitive domains comprises receiving information relating to a life history of the client, interests of the client, and family information of the client.

7. The method of claim 1, wherein determining a program of intervention activities associated with each cognitive and non-cognitive areas comprises manually selecting one or more intervention activities from a plurality of available intervention activities.

8. A method of enhancing neurophysiological processes of a client, said method comprising:
receiving a program of intervention activities including a plurality of intervention activities, each intervention activity associated with at least one of a cognitive and a non-cognitive area;
administering at least a portion of the program of intervention activities to the client, the portion based on a level of cooperation of the client;
recording an evaluation of the success of the administering based on predetermined completion thresholds; and
reporting a progress of the client over time with respect to the recorded evaluations for each of the domains, cognitive subdomains, and non-cognitive areas evaluated.

9. The method of claim 8, wherein receiving a program of intervention activities comprises receiving a program of intervention activities that have been modified since the last administering based on the recorded evaluation.

10. The method of claim 8, wherein receiving a program of intervention activities comprises receiving a program of intervention activities associated with at least one of a cognitive domain, a coping domain, a social domain, a recreational domain, an exercise domain, a sensory domain, and a diet domain of the client.

11. The method of claim 10, wherein receiving a program of intervention activities associated with a cognitive domain comprises receiving a program of intervention activities associated with a memory subdomain, an executive function subdomain, an attention subdomain, a visual-spatial subdomain, and a language subdomain of the client.

12. The method of claim 8, wherein recording an evaluation of the success of the administering comprises recording the evaluation by a staff personnel administering the evaluation.

13. The method of claim 12, wherein recording the evaluation by a staff personnel administering the evaluation comprises recording a subjective score indicating the success using a predetermined scale.

14. A method of administering a program of intervention activities to reduce progress of a chronic progressive neurodegenerative condition, said method comprising:
receiving an evaluation of a client, the evaluation including at least one of a new client evaluation and a progress evaluation;
receiving a plurality of available intervention activities from a library of approved intervention activities;
generating a program of intervention activities for administration to the client based on the at least one of a new client evaluation and a progress evaluation, and the plurality of available intervention activities, the program of intervention activities including a plurality of the available intervention activities, each intervention activity in the plurality of the available intervention activities being associated with a domain, a cognitive subdomain, and/or a non-cognitive area; and
transmitting the program of intervention activities to the staff personnel for administration to the client and reporting the progress of the client.

15. The method of claim 14, wherein generating a program of intervention activities comprises generating a program of intervention activities associated with a cognitive domain, a coping domain, a social domain, a recreational domain, an exercise domain, a sensory domain, and a diet domain of the client.

16. The method of claim 15, wherein generating a program of intervention activities associated with a cognitive domain comprises generating a program of intervention activities associated with a memory subdomain, an executive function subdomain, an attention subdomain, a visual-spatial subdomain, and a language subdomain.

17. The method of claim 14, wherein generating a program of intervention activities comprises generating a program of intervention activities associated with at least one of behavior, motivation, persistence, dependability, self-confidence global functioning and activities of daily living of the client.

18. The method of claim 14, wherein generating a program of intervention activities comprises generating instructions for conducting each of the intervention activities, the instructions including performance thresholds and measurements of success.

19. The method of claim 18, wherein receiving an evaluation of a client comprises receiving an evaluation of a client that includes a subjective score indicating a success of performing each of the intervention activities using a predetermined scale.
